(19) United States
(12) Patent Application Publication
(10) Pub. No.: US 2026/0293894 A1
(43) Pub. Date: Oct. 1, 2026

(54) FUSED PYRROLIDINE-2-ONE HERBICIDAL COMPOUNDS

(71) Applicant: SYNGENTA CROP PROTECTION AG, Basel (CH)

(72) Inventors: James Alan MORRIS, Bracknell Berkshire (GB); Kenneth Bruce LING, Bracknell Berkshire (GB); Andrew George DALLING, Bracknell Berkshire (GB)

(73) Assignee: SYNGENTA CROP PROTECTION AG, Basel (CH)

(21) Appl. No.: 19/473,027

(22) PCT Filed: Apr. 5, 2024
(86) PCT No.: PCT/EP2024/059284

§ 371 Date: Oct. 6, 2025

(30) Foreign Application Priority Data
Apr. 6, 2023 (GB) 2305125.3

(51) Int. Cl. A01N 43/38; A01P 13/00; C07D 209/34; C07D 209/52

(52) U.S. Cl. CPC A01N 43/38; A01P 13/00; C07D 209/34; C07D 209/52

(57) ABSTRACT

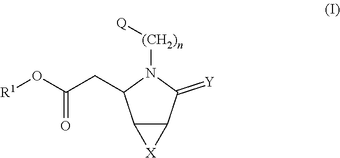

The present invention relates to compounds of Formula (I), or an agronomically acceptable stereoisomers and/or salt of said compounds wherein Q, X, Y, R¹ and n are as defined herein. The invention further relates to herbicidal compositions which comprise a compound of Formula (I) and to the use of compounds of Formula (I) for controlling weeds, in particular in crops of useful plants.

The present invention relates to novel herbicidal compounds, to processes for their preparation, to herbicidal compositions which comprise the novel compounds, and to their use for controlling weeds, in particular in crops of useful plants, or for inhibiting plant growth.

Herbicidal pyrrolidine-2-ones are known from EP-A-0004107, U.S. Pat. No. 5,352,655 and WO2022/200208. Thus, according to the present invention there is provided a compound of Formula (I)

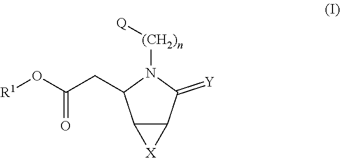

wherein
X=−(CR²R³)ₘ—;
Y=O or S;
Q is phenyl optionally substituted by 1-4 R⁴ substituents;
R¹ is hydrogen or C₁-C₆ alkyl
R² and R³ are independently selected from the group consisting of hydrogen, methyl, fluoro and chloro;
R⁴ is halogen, cyano, C₁-C₆-alkyl, C₁-C₆alkoxy-, C₁-C₆haloalkyl, C₁-C₆-haloalkoxy- and —S(O)ₚC₁-C₆alkyl;
m=1, 2, 3 or 4;
n=1 or 2; and
p=0, 1 or 2.

C₁-C₆alkyl-includes, for example, methyl (Me, CH₃), ethyl (Et, C₂H₅), n-propyl (n-Pr), isopropyl (i-Pr), n-butyl (n-Bu), isobutyl (i-Bu), sec-butyl and tert-butyl (t-Bu). C₁-C₂alkyl is methyl (Me, CH₃) or ethyl (Et, C₂H₅).

Halogen (or halo) includes, for example, fluorine, chlorine, bromine or iodine. The same correspondingly applies to halogen in the context of other definitions, such as haloalkyl.

C₁-C₆haloalkyl-includes, for example, fluoromethyl, difluoromethyl, trifluoromethyl, chloromethyl, dichloromethyl, trichloromethyl, 2,2,2-trifluoroethyl, 2-fluoroethyl, 2-chloroethyl, pentafluoroethyl, 1,1-difluoro-2,2,2-trichloroethyl, 2,2,3,3-tetrafluoropropyl and 2,2,2-trichloroethyl and heptafluoro-n-propyl. C₁-C₂haloalkyl is, for example, fluoromethyl, difluoromethyl, trifluoromethyl, chloromethyl, dichloromethyl, trichloromethyl, 2,2,2-trifluoroethyl, 2-fluoroethyl, 2-chloroethyl, pentafluoroethyl, or 1,1-difluoro-2,2,2-trichloroethyl.

C₁-C₆alkoxy includes methoxy and ethoxy.

C₁-C₄haloalkoxy-includes, for example, fluoromethoxy, difluoromethoxy, trifluoromethoxy, 2,2,2-trifluoroethoxy, 1,1,2,2-tetrafluoroethoxy, 2-fluoroethoxy, 2-chloroethoxy, 2,2-difluoroethoxy or 2,2,2-trichloroethoxy, preferably difluoromethoxy, 2-chloroethoxy or trifluoromethoxy.

C₁-C₆alkyl-S-(alkylthio) includes, for example, methylthio, ethylthio, propylthio, isopropylthio, n-butylthio, isobutylthio, sec-butylthio or tert-butylthio, preferably methylthio or ethylthio.

C₁-C₆alkyl-S(O)-(alkylsulfinyl) includes, for example, methylsulfinyl, ethylsulfinyl, propylsulfinyl, isopropylsulfinyl, n-butylsulfinyl, isobutylsulfinyl, sec-butylsulfinyl or tert-butylsulfinyl, preferably methylsulfinyl or ethylsulfinyl.

C₁-C₆alkyl-S(O)₂-(alkylsulfonyl) includes, for example, methylsulfonyl, ethylsulfonyl, propylsulfonyl, isopropylsulfonyl, n-butylsulfonyl, isobutylsulfonyl, sec-butylsulfonyl or tert-butylsulfonyl, preferably methylsulfonyl or ethylsulfonyl.

In one embodiment of the present invention Y is S. In a preferred embodiment of the present invention Y is O.

In a preferred embodiment of the present invention wherein X is —(CR²R³)—. In a more preferred embodiment of the present invention X is —(CH₂)—.

In one embodiment of the present invention, Q in unsubstituted phenyl. In a preferred embodiment of the present invention Q is substituted by one, two or three (preferably two or three) R⁴. In a more preferred embodiment of the present invention, Q is 2,3-difluorophenyl, 2,3,4-trifluorophenyl or 2,3,5-trifluorophenyl.

In a preferred embodiment of the present invention, R¹ is hydrogen or methyl.

Compounds of Formula (I) may contain asymmetric centres and may be present as a single enantiomer, pairs of enantiomers in any proportion or, where more than one asymmetric centre are present, contain diastereoisomers in all possible ratios. Typically, one of the stereoisomers has enhanced biological activity compared to the other possibilities. For example, in the context of the present invention compounds of Formula (I) wherein X is CH₂ may exist in the following alternative forms, wherein the relative stereochemistry between C(1) and C(2) may alternatively be cis- or trans-, and each diastereoisomer is further comprised of a pair of individual enantiomers with opposite absolute configuration, for example the enantiomeric pair (1a) (1R,2R,5S) and (1b) (1S,2S,5R), and the enantiomeric pair (1c) (1R,2S,5S) and (1d) (1S,2R,5R):

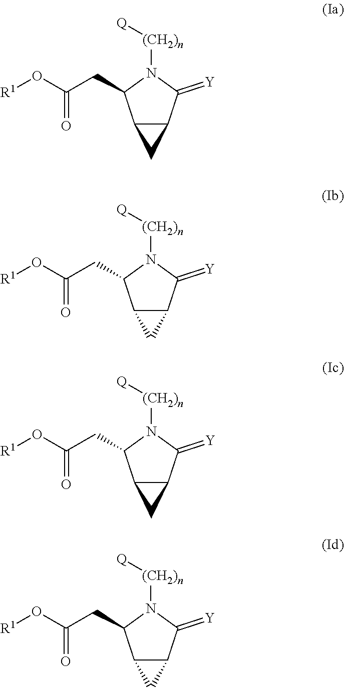

The skilled person is aware of art-recognised methods for separating the various stereoisomers of compounds of Formula (I).

The present invention also provides agronomically acceptable salts of compounds of Formula (I). Salts that the compounds of Formula (I) may form with amines, including primary, secondary and tertiary amines (for example ammonia, dimethylamine and triethylamine), alkali metal and alkaline earth metal bases, transition metals or quaternary ammonium bases are preferred.

The compounds of Formula (I) according to the invention can be used as herbicides by themselves, but they are generally formulated into herbicidal compositions using formulation adjuvants, such as carriers, solvents and surface-active agents (SAA). Thus, the present invention further provides a herbicidal composition comprising a herbicidal compound according to any one of the previous claims and an agriculturally acceptable formulation adjuvant. The composition can be in the form of concentrates which are diluted prior to use, although ready-to-use compositions can also be made. The final dilution is usually made with water, but can be made instead of, or in addition to, water, with, for example, liquid fertilisers, micronutrients, biological organisms, oil or solvents.

The herbicidal compositions generally comprise from 0.1 to 99% by weight, especially from 0.1 to 95% by weight, compounds of Formula I and from 1 to 99.9% by weight of a formulation adjuvant which preferably includes from 0 to 25% by weight of a surface-active substance.

The compositions can be chosen from a number of formulation types. These include an emulsion concentrate (EC), a suspension concentrate (SC), a suspo-emulsion (SE), a capsule suspension (CS), a water dispersible granule (WG), an emulsifiable granule (EG), an emulsion, water in oil (EO), an emulsion, oil in water (EW), a micro-emulsion (ME), an oil dispersion (OD), an oil miscible flowable (OF), an oil miscible liquid (OL), a soluble concentrate (SL), an ultra-low volume suspension (SU), an ultra-low volume liquid (UL), a technical concentrate (TK), a dispersible concentrate (DC), a soluble powder (SP), a wettable powder (WP) and a soluble granule (SG). The formulation type chosen in any instance will depend upon the particular purpose envisaged and the physical, chemical and biological properties of the compound of Formula (I).

Soluble powders (SP) may be prepared by mixing a compound of Formula (I) with one or more water-soluble inorganic salts (such as sodium bicarbonate, sodium carbonate or magnesium sulphate) or one or more water-soluble organic solids (such as a polysaccharide) and, optionally, one or more wetting agents, one or more dispersing agents or a mixture of said agents to improve water dispersibility/solubility. The mixture is then ground to a fine powder. Similar compositions may also be granulated to form water soluble granules (SG).

Wettable powders (WP) may be prepared by mixing a compound of Formula (I) with one or more solid diluents or carriers, one or more wetting agents and, preferably, one or more dispersing agents and, optionally, one or more suspending agents to facilitate the dispersion in liquids. The mixture is then ground to a fine powder. Similar compositions may also be granulated to form water dispersible granules (WG).

Granules (GR) may be formed either by granulating a mixture of a compound of Formula (I) and one or more powdered solid diluents or carriers, or from pre-formed blank granules by absorbing a compound of Formula (I) (or a solution thereof, in a suitable agent) in a porous granular material (such as pumice, attapulgite clays, fuller's earth, kieselguhr, diatomaceous earths or ground corn cobs) or by adsorbing a compound of Formula (I) (or a solution thereof, in a suitable agent) on to a hard core material (such as sands, silicates, mineral carbonates, sulphates or phosphates) and drying if necessary. Agents which are commonly used to aid absorption or adsorption include solvents (such as aliphatic and aromatic petroleum solvents, alcohols, ethers, ketones and esters) and sticking agents (such as polyvinyl acetates, polyvinyl alcohols, dextrins, sugars and vegetable oils). One or more other additives may also be included in granules (for example an emulsifying agent, wetting agent or dispersing agent).

Dispersible Concentrates (DC) may be prepared by dissolving a compound of Formula (I) in water or an organic solvent, such as a ketone, alcohol or glycol ether. These solutions may contain a surface-active agent (for example to improve water dilution or prevent crystallisation in a spray tank).

Emulsifiable concentrates (EC) or oil-in-water emulsions (EW) may be prepared by dissolving a compound of Formula (I) in an organic solvent (optionally containing one or more wetting agents, one or more emulsifying agents or a mixture of said agents). Suitable organic solvents for use in ECs include aromatic hydrocarbons (such as alkylbenzenes or alkylnaphthalenes, exemplified by SOLVESSO 100, SOLVESSO 150 and SOLVESSO 200; SOLVESSO is a Registered Trade Mark), ketones (such as cyclohexanone or methylcyclohexanone) and alcohols (such as benzyl alcohol, furfuryl alcohol or butanol), N-alkylpyrrolidones (such as N-methylpyrrolidone or N-octylpyrrolidone), dimethyl amides of fatty acids (such as C₈-C₁₀ fatty acid dimethylamide) and chlorinated hydrocarbons. An EC product may spontaneously emulsify on addition to water, to produce an emulsion with sufficient stability to allow spray application through appropriate equipment.

Preparation of an EW involves obtaining a compound of Formula (I) either as a liquid (if it is not a liquid at room temperature, it may be melted at a reasonable temperature, typically below 70° C.) or in solution (by dissolving it in an appropriate solvent) and then emulsifying the resultant liquid or solution into water containing one or more SAAs, under high shear, to produce an emulsion. Suitable solvents for use in EWs include vegetable oils, chlorinated hydrocarbons (such as chlorobenzenes), aromatic solvents (such as alkylbenzenes or alkylnaphthalenes) and other appropriate organic solvents which have a low solubility in water.

Microemulsions (ME) may be prepared by mixing water with a blend of one or more solvents with one or more SAAs, to produce spontaneously a thermodynamically stable isotropic liquid formulation. A compound of Formula (I) is present initially in either the water or the solvent/SAA blend. Suitable solvents for use in MEs include those hereinbefore described for use in in ECs or in EWs. An ME may be either an oil-in-water or a water-in-oil system (which system is present may be determined by conductivity measurements) and may be suitable for mixing water-soluble and oil-soluble pesticides in the same formulation. An ME is suitable for dilution into water, either remaining as a microemulsion or forming a conventional oil-in-water emulsion.

Suspension concentrates (SC) may comprise aqueous or non-aqueous suspensions of finely divided insoluble solid particles of a compound of Formula (I). SCs may be prepared by ball or bead milling the solid compound of Formula (I) in a suitable medium, optionally with one or more dispersing agents, to produce a fine particle suspension of the compound. One or more wetting agents may be included in the composition and a suspending agent may be included to reduce the rate at which the particles settle. Alternatively, a compound of Formula (I) may be dry milled and added to water, containing agents hereinbefore described, to produce the desired end product.

Aerosol formulations comprise a compound of Formula (I) and a suitable propellant (for example n-butane). A compound of Formula (I) may also be dissolved or dispersed in a suitable medium (for example water or a water miscible liquid, such as n-propanol) to provide compositions for use in non-pressurised, hand-actuated spray pumps.

Capsule suspensions (CS) may be prepared in a manner similar to the preparation of EW formulations but with an additional polymerisation stage such that an aqueous dispersion of oil droplets is obtained, in which each oil droplet is encapsulated by a polymeric shell and contains a compound of Formula (I) and, optionally, a carrier or diluent therefor. The polymeric shell may be produced by either an interfacial polycondensation reaction or by a coacervation procedure. The compositions may provide for controlled release of the compound of Formula (I) and they may be used for seed treatment. A compound of Formula (I) may also be formulated in a biodegradable polymeric matrix to provide a slow, controlled release of the compound.

The composition may include one or more additives to improve the biological performance of the composition, for example by improving wetting, retention or distribution on surfaces; resistance to rain on treated surfaces; or uptake or mobility of a compound of Formula (I). Such additives include surface active agents (SAAs), spray additives based on oils, for example certain mineral oils or natural plant oils (such as soy bean and rape seed oil), modified plant oils such as methylated rape seed oil (MRSO), and blends of these with other bio-enhancing adjuvants (ingredients which may aid or modify the action of a compound of Formula (I).

Wetting agents, dispersing agents and emulsifying agents may be SAAs of the cationic, anionic, amphoteric or non-ionic type.

Suitable SAAs of the cationic type include quaternary ammonium compounds (for example cetyltrimethyl ammonium bromide), imidazolines and amine salts.

Suitable anionic SAAs include alkali metals salts of fatty acids, salts of aliphatic monoesters of sulphuric acid (for example sodium lauryl sulphate), salts of sulphonated aromatic compounds (for example sodium dodecylbenzenesulphonate, calcium dodecylbenzenesulphonate, butylnaphthalene sulphonate and mixtures of sodium di-isopropyl- and tri-isopropyl-naphthalene sulphonates), ether sulphates, alcohol ether sulphates (for example sodium laureth-3-sulphate), ether carboxylates (for example sodium laureth-3-carboxylate), phosphate esters (products from the reaction between one or more fatty alcohols and phosphoric acid (predominately mono-esters) or phosphorus pentoxide (predominately di-esters), for example the reaction between lauryl alcohol and tetraphosphoric acid; additionally these products may be ethoxylated), sulphosuccinamates, paraffin or olefine sulphonates, taurates, lignosulphonates and phosphates/sulphates of tristyrylphenols.

Suitable SAAs of the amphoteric type include betaines, propionates and glycinates.

Suitable SAAs of the non-ionic type include condensation products of alkylene oxides, such as ethylene oxide, propylene oxide, butylene oxide or mixtures thereof, with fatty alcohols (such as oleyl alcohol or cetyl alcohol) or with alkylphenols (such as octylphenol, nonylphenol or octylcresol); partial esters derived from long chain fatty acids or hexitol anhydrides; condensation products of said partial esters with ethylene oxide; block polymers (comprising ethylene oxide and propylene oxide); alkanolamides; simple esters (for example fatty acid polyethylene glycol esters); amine oxides (for example lauryl dimethyl amine oxide); lecithins and sorbitans and esters thereof, alkyl polyglycosides and tristyrylphenols.

Suitable suspending agents include hydrophilic colloids (such as polysaccharides, polyvinylpyrrolidone or sodium carboxymethylcellulose) and swelling clays (such as bentonite or attapulgite).

The compounds of present invention can also be used in mixture with one or more additional herbicides and/or plant growth regulators. Examples of such additional herbicides or plant growth regulators include acetochlor, acifluorfen (including acifluorfen-sodium), aclonifen, ametryn, amicarbazone, aminopyralid, aminotriazole, atrazine, beflubutamid-M, benquitrione, bensulfuron (including bensulfuron-methyl), bentazone, bicyclopyrone, bilanafos, bipyrazone, bispyribac-sodium, bixlozone, broclozone, bromacil, bromoxynil, butachlor, butafenacil, carfentrazone (including carfentrazone-ethyl), cloransulam (including cloransulam-methyl), chlorimuron (including chlorimuron-ethyl), chlorotoluron, chlorsulfuron, cinmethylin, clacyfos, clethodim, clodinafop (including clodinafop-propargyl), clomazone, clopyralid, cyclopyranil, cyclopyrimorate, cyclosulfamuron, cyhalofop (including cyhalofop-butyl), 2,4-D (including the choline salt and 2-ethylhexyl ester thereof), 2,4-DB, desmedipham, dicamba (including the aluminium, aminopropyl, bis-aminopropylmethyl, choline, dichloroprop, diglycolamine, dimethylamine, dimethylammonium, potassium and sodium salts thereof) diclosulam, diflufenican, diflufenzopyr, dimethachlor, dimethenamid-P, dioxopyritrione, diquat dibromide, diuron, epyrifenacil, ethalfluralin, ethofumesate, fenoxaprop (including fenoxaprop-P-ethyl), fenoxasulfone, fenpyrazone, fenquinotrione, fentrazamide, flazasulfuron, florasulam, florpyrauxifen (including florpyrauxifen-benzyl), fluazifop (including fluazifop-P-butyl), flucarbazone (including flucarbazone-sodium), fluchloraminopyr (including fluchloramino-tefuryl), flufenacet, flufenoximacil, flumetsulam, flumioxazin, fluometuron, fomesafen flupyrsulfuron (including flupyrsulfuron-methyl-sodium), fluroxypyr (including fluroxypyr-meptyl), flusulfinam, fomesafen, foramsulfuron, glufosinate (including L-glufosinate and the ammonium salts of both), glyphosate (including the diammonium, isopropylammonium and potassium salts thereof), halauxifen (including halauxifen-methyl), haloxyfop (including haloxyfop-methyl), hexazinone, hydantocidin, icafolin (including icafolin-methyl), imazamox (including R-imazamox), imazapic, imazapyr, imazethapyr, indaziflam, indolauxipyr (including indolauxipyr-cyanomethyl), iodosulfuron (including iodosulfuron-methyl-sodium), iofensulfuron (including iofensulfuron-sodium), ioxynil, iptriazopyrid, isoproturon, isoxaflutole, lancotrione, MCPA, MCPB, mecoprop-P, mesosulfuron (including mesosulfuron-methyl), mesotrione, metamitron, metazachlor, methiozolin, metolachlor, metosulam, metribuzin, metsulfuron, napropamide, nicosulfuron, norflurazon, oxadiazon, oxasulfuron, oxyfluorfen, paraquat dichloride, pendimethalin, penoxsulam, phenmedipham, picloram, pinoxaden, pretilachlor, primisulfuron-methyl, prometryne, propanil, propaquizafop, propyrisulfuron, propyzamide, prosulfocarb, prosulfuron, pyraclonil, pyraflufen (including pyraflufen-ethyl), pyraquinate, pyrasulfotole, pyridate, pyriftalid, pyriflubenzoxim, pyrimisulfan, pyroxasulfone, pyroxsulam, quinclorac, quinmerac, quizalofop (including quizalofop-P-ethyl and quizalofop-P-tefuryl), rimisoxafen, rimsulfuron, saflufenacil, sethoxydim, simazine, S-metalochlor, sulfentrazone, sulfosulfuron, tebuthiuron, tefuryltrione, tembotrione, terbuthylazine, terbutryn, tetflupyrolimet, thiencarbazone, thifensulfuron, tiafenacil, tolpyralate, topramezone, tralkoxydim, triafamone, triallate, triasulfuron, tribenuron (including tribenuron-methyl), triclopyr, trifloxysulfuron (including trifloxysulfuron-sodium), trifludimoxazin, trifluralin, triflusulfuron, tripyrasulfone, 3-(2-chloro-4-fluoro-5-(3-methyl-2,6-dioxo-4-trifluoromethyl-3,6-dihydropyrimidin-1 (2H)-yl)phenyl)-5-methyl-4,5-dihydroisoxazole-5-carboxylic acid ethyl ester, 4-hydroxy-1-methoxy-5-methyl-3-[4-(trifluoromethyl)-2-pyridyl]imidazolidin-2-one, 4-hydroxy-1,5-dimethyl-3-[4-(trifluoromethyl)-2-pyridyl]imidazolidin-2-one, 5-ethoxy-4-hydroxy-1-methyl-3-[4-(trifluoromethyl)-2-pyridyl]imidazolidin-2-one, 4-hydroxy-1-methyl-3-[4-(trifluoromethyl)-2-pyridyl]imidazolidin-2-one, 4-hydroxy-1,5-dimethyl-3-[1-methyl-5-(trifluoromethyl) pyrazol-3-yl]imidazolidin-2-one, (4R) 1-(5-tert-butylisoxazol-3-yl)-4-ethoxy-5-hydroxy-3-methyl-imidazolidin-2-one, 3-ethylsulfanyl-N-(1,3,4-oxadiazol-2-yl)-5-(trifluoromethyl)-[1,2,4]triazolo[4,3-a]pyridine-8-carboxamide, 3-(isopropylsulfanylmethyl)-N-(5-methyl-1,3,4-oxadiazol-2-yl)-5-(trifluoromethyl)-[1,2,4]triazolo[4,3-a]pyridine-8-carboxamide, ethyl-2-[[3-[[3-chloro-5-fluoro-6-[3-methyl-2,6-dioxo-4-(trifluoromethyl)pyrimidin-1-yl]-2-pyridyl]oxy]acetate, methyl 2-[2-[2-bromo-4-fluoro-5-[3-methyl-2,6-dioxo-4-(trifluoromethyl)pyrimidin-1-yl]phenoxylphenoxy]-2-methoxy-acetate, 6-chloro-4-(2,7-dimethyl-1-naphthyl)-5-hydroxy-2-methyl-pyridazin-3-one, (2-fluorophenyl)methyl 6-amino-5-chloro-2-(4-chloro-2-fluoro-3-methoxy-phenyl)pyrimidine-4-carboxylate, 6-amino-5-chloro-2-(4-chloro-2-fluoro-3-methoxy-phenyl)pyrimidine-4-carboxylic acid, and methyl 3-[2-chloro-5-[3,6-dihydro-3-methyl-2,6-dioxo-4-(trifluoromethyl)-1 (2H)-pyrimidinyl]-4-fluorophenyl]-3a,4,5,6-tetrahydro-6-methyl-6aH-cyclopent[d]isoxazole-6a-carboxylate.

The mixing partners of the compound of Formula (I) may also be in the form of esters or salts, as mentioned e.g. in The Pesticide Manual, Sixteenth Edition, British Crop Protection Council, 2012.

The compound of Formula (I) can also be used in mixtures with other agrochemicals such as fungicides, nematicides or insecticides, examples of which are given in The Pesticide Manual.

The mixing ratio of the compound of Formula (I) to the mixing partner is preferably from 1:100 to 1000:1.

The mixtures can advantageously be used in the above-mentioned formulations (in which case “active ingredient” relates to the respective mixture of compound of Formula (I) with the mixing partner).

The compounds or mixtures of the present invention can also be used in combination with one or more herbicide safeners. Examples of such safeners include benoxacor, cloquintocet (including cloquintocet-mexyl), cyprosulfamide, dichlormid, fenchlorazole (including fenchlorazole-ethyl), fenclorim, fluxofenim, furilazole, isoxadifen (including isoxadifen-ethyl), mefenpyr (including mefenpyr-diethyl), metcamifen and oxabetrinil.

Particularly preferred are mixtures of a compound of Formula (I) with cyprosulfamide, isoxadifen-ethyl, cloquintocet-mexyl and/or metcamifen.

The safeners of the compound of Formula (I) may also be in the form of esters or salts, as mentioned e.g. in The Pesticide Manual, 16^{th} Edition (BCPC), 2012. The reference to cloquintocet-mexyl also applies to a lithium, sodium, potassium, calcium, magnesium, aluminium, iron, ammonium, quaternary ammonium, sulfonium or phos-phonium salt thereof as disclosed in WO 02/34048.

Preferably the mixing ratio of compound of Formula (I) to safener is from 100:1 to 1:10, especially from 20:1 to 1:1.

The present invention still further provides a method of controlling weeds at a locus said method comprising application to the locus of a weed controlling amount of a composition comprising a compound of Formula (I). Moreover, the present invention may further provide a method of selectively controlling weeds at a locus comprising crop plants and weeds, wherein the method comprises application to the locus of a weed controlling amount of a composition according to the present invention. ‘Controlling’ means killing, reducing or retarding growth or preventing or reducing germination. It is noted that the compounds of the present invention show a much-improved selectivity compared to know, structurally similar compounds. Generally the plants to be controlled are unwanted plants (weeds). ‘Locus’ means the area in which the plants are growing or will grow. The application may be applied to the locus pre-emergence and/or postemergence of the crop plant. Some crop plants may be inherently tolerant to herbicidal effects of compounds of Formula (I). Preferred crop plants include maize, wheat, barley soybean and rice.

The rates of application of compounds of Formula I may vary within wide limits and depend on the nature of the soil, the method of application (pre- or post-emergence; seed dressing; application to the seed furrow; no tillage application etc.), the crop plant, the weed(s) to be controlled, the prevailing climatic conditions, and other factors governed by the method of application, the time of application and the target crop. The compounds of Formula I according to the invention are generally applied at a rate of from 10 to 2500 g/ha, especially from 25 to 1000 g/ha, more especially from 25 to 250 g/ha.

The application is generally made by spraying the composition, typically by tractor mounted sprayer for large areas, but other methods such as dusting (for powders), drip or drench can also be used.

Crop plants are to be understood as also including those crop plants which have been rendered tolerant to other herbicides or classes of herbicides (e.g. ALS-, GS-, EPSPS-, PPO-, HPPD-, -PDS and ACCase-inhibitors) by conventional methods of breeding or by genetic engineering. An example of a crop that has been rendered tolerant to imidazolinones, e.g. imazamox, by conventional methods of breeding is Clearfield® summer rape (canola). Examples of crops that have been rendered tolerant to herbicides by genetic engineering methods include e.g. glyphosate- and glufosinate-resistant maize varieties commercially available under the trade names RoundupReady® and LibertyLink®. The compounds of the present invention can also be used in conjunction with crops that are tolerant to SDPS-inhibiting herbicides, such as those taught in WO2020/236790.

Crop plants are also to be understood as being those which have been rendered resistant to harmful insects by genetic engineering methods, for example Bt maize (resistant to European corn borer), Bt cotton (resistant to cotton boll weevil) and also Bt potatoes (resistant to Colorado beetle). Examples of Bt maize are the Bt 176 maize hybrids of NK® (Syngenta Seeds). The Bt toxin is a protein that is formed naturally by Bacillus thuringiensis soil bacteria. Examples of toxins, or transgenic plants able to synthesise such toxins, are described in EP-A-451 878, EP-A-374 753, WO 93/07278, WO 95/34656, WO 03/052073 and EP-A-427 529. Examples of transgenic plants comprising one or more genes that code for an insecticidal resistance and express one or more toxins are KnockOut® (maize), Yield Gard® (maize), NuCOTIN33B® (cotton), Bollgard® (cotton), NewLeaf® (potatoes), NatureGard® and Protexcta®. Plant crops or seed material thereof can be both resistant to herbicides and, at the same time, resistant to insect feeding (“stacked” transgenic events). For example, seed can have the ability to express an insecticidal Cry3 protein while at the same time being tolerant to glyphosate.

Crop plants are also to be understood to include those which are obtained by conventional methods of breeding or genetic engineering and contain so-called output traits (e.g. improved storage stability, higher nutritional value and improved flavour).

The compositions can be used to control unwanted plants (collectively, ‘weeds’). The weeds to be controlled may be both monocotyledonous species, for example Agrostis, Alopecurus, Avena, Brachiaria, Bromus, Cenchrus, Cyperus, Digitaria, Echinochloa, Eleusine, Lolium, Monochoria, Rottboellia, Sagittaria, Scirpus, Setaria and Sorghum, and dicotyledonous species, for example Abutilon, Amaranthus, Ambrosia, Chenopodium, Chrysanthemum, Conyza, Galium, Ipomoea, Nasturtium, Sida, Sinapis, Solanum, Stellaria, Veronica, Viola and Xanthium. The compounds and compositions of the present invention are particularly suited to control Digitaria, Echinochloa, Eleusine, Lolium and Setaria species.

In a further aspect of the present invention there is provided the use of a compound of Formula (I) as defined herein as a herbicide.

### Processes for Preparation of Compounds of Formula (I)

The compounds of formula (II), are obtained by Horner-Wadsworth-Emmons reaction of compounds of formula (III) with phosphonate esters of formula (IV), wherein R⁵ is a C₁-C₆ alkyl chain (e.g. methyl or ethyl), in the presence of a base (e.g. sodium hydride, sodium hexamethyldisilylazide, n-butyllithium or sodium tert-butoxide), preferably in a suitable solvent (e.g., tetrahydrofuran, 1,2-dimethoxyethane, diethyl ether, N,N-dimethylformamide or dimethylsulfoxide), preferably at temperatures between −78° C. and 80° C.

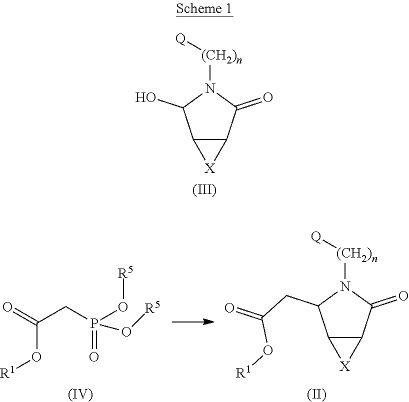

The compounds of formula (III) are prepared by reduction of compounds of formula (V) with a suitable reducing agent (e.g. sodium borohydride, lithium borohydride or diisobutylaluminium hydride), in a suitable solvent (e.g. methanol, ethanol or tetrahydrofuran) at temperatures between −78° C. and 30° C., and optionally in the presence of a co-solvent such as dichloromethane.

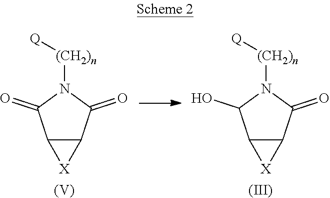

The compounds of formula (V) are prepared by addition of anhydrides (VI) with amines (VII), or salts thereof, in a suitable solvent (e.g. polyethylene glycol 400, toluene, N,N-dimethylformamide or dimethylsulfoxide), at temperatures between 20° C. and 140° C., optionally in the presence of an acid such as acetic acid, or a base such as triethylamine.

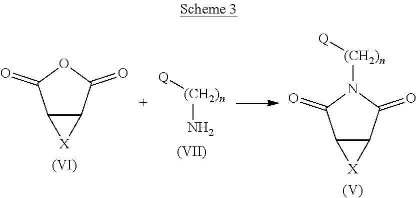

The compounds of formula (VI) are commercially available or can be prepared from diacids (VIII), in the presence of an acylating agent (e.g. acetic anhydride, trifluoroacetic anhydride or acetyl chloride) at temperatures between 20° C. and 140° C., optionally in a suitable solvent (e.g. tetrahydrofuran, toluene, dichloromethane, isopropyl acetate), and optionally in the presence of a base (e.g. triethylamine, sodium acetate or sodium hydroxide) or an acid (e.g. acetic acid), or alternatively under conditions described in the literature for a condensation reaction such as phosphorous pentoxide in a suitable solvent (e.g. dichloromethane).

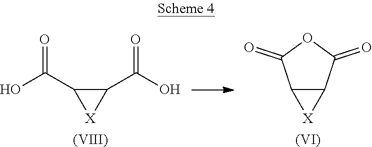

The compounds of formula (IX), are obtained by thionation reaction of compounds of formula (II), with a thionation reagent (e.g. Lawesson's reagent or P₂S₅), optionally in the presence of a base (e.g. sodium hydrogen carbonate or triethylamine), preferably in a suitable solvent (e.g., tetrahydrofuran, toluene, acetonitrile or dichloromethane), preferably at temperatures between 20° C. and 120° C.

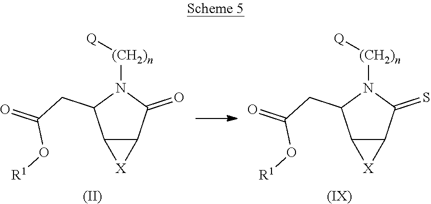

Alternatively, compounds of formula (X) wherein the relative stereochemistry is cis- with respect to the stereocentres at C(1) and C(5), can be prepared by reduction of compounds of formula (XI). Suitable conditions include reduction using a silane (e.g. triethylsilane), in combination with an acid (e.g. trifluoroacetic acid), preferably in the presence of a suitable solvent (e.g. dichloromethane), preferably at temperatures between 0° C. and 40° C.

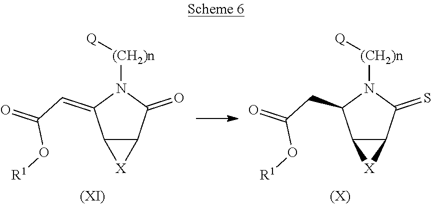

Compounds of formula (XI) can be prepared by cyclisation of compounds of formula (XII), preferably in the presence of an acid (e.g. p-toluenesulfonic acid), preferably in the presence of a solvent (e.g. 1,2-dichloroethane), preferably at temperatures between 20° C. and 120° C.

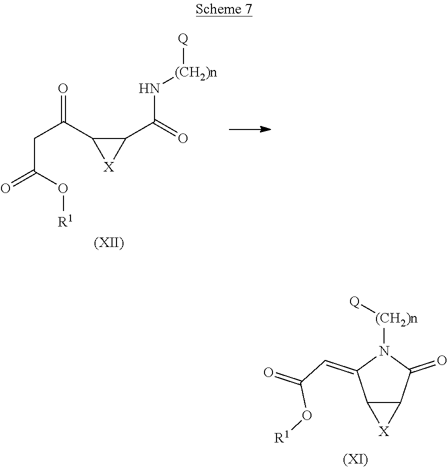

Compounds of formula (XII) can be prepared from compounds of formula (XIII) by reaction with an amine of formula (VII), preferably in a solvent (e.g. toluene), preferably at temperatures between 20° C. and 120° C.

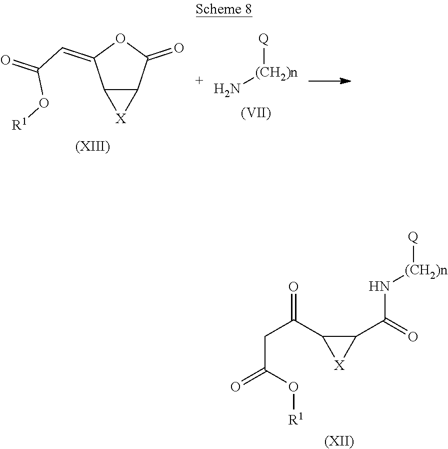

Compounds of formula (XIII) can be prepared according to methods described in the literature e.g. Australian Journal of Chemistry, 1982, vol. 35, #9, p. 1903-1911.

Where compounds are prepared as mixture of diastereoisomers, they can be separated by techniques that are commonly employed by one skilled in the art, for example flash column chromatography or preparative HPLC. Where compounds are prepared as racemic mixtures, they can be separated by techniques that are commonly employed by one skilled in the art, for example preparative HPLC using a chiral stationary phase or chiral resolution (e.g. via formation of diastereoisomeric salts).

The following non-limiting examples provide specific synthesis methods for representative compounds of the present invention, as referred to in the Tables below.

### LCMS Methods:

### Method 1:

Spectra were recorded on a Mass Spectrometer from Waters (SQD2 or QDA Single quadrupole mass spectrometer) equipped with an electrospray source (Polarity: Positive and Negative Polarity Switch), Capillary: 0.8-3.00 kV, Cone range: 25 Source Temperature: 120-150° C., Desolvation Temperature: 500-600° C., Cone Gas Flow: 50 L/h, Desolvation Gas Flow: 1000 L/h, Mass range: 110 to 850 Da) and an Acquity UPLC from Waters: Quaternary solvent manager, heated column compartment, diode-array detector. Column: Acquity UPLC HSS T3 C18, 1.8 μm, 30×2.1 mm, Temp: 40° C., DAD Wavelength range (nm): 200 to 400, Solvent Gradient: A=water+5% Acetonitrile+0.1% HCOOH, B=Acetonitrile+0.05% HCOOH: gradient: 0 min 10% B; 0-0.2 min 10-50% B; 0.2-0.6 min 50-100% B; 0.6-1.3 min 100% B; 1.3-1.4 min 100-10% B; 1.4-1.6 min 10% B; Flow (mL/min) 0.6.

### Example 1: 2-[(1R*,2S*,5S*)-3-[(2,3-difluorophenyl)methyl]-4-oxo-3-azabicyclo[3.1.0]hexan-2-yl]acetic acid (Compound A.04)

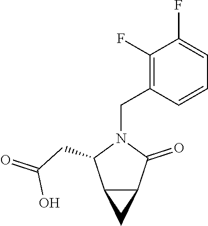 [SMILES: O=C(O)C[C@H]1[C@@H]2C[C@@H]2C(=O)N1Cc1cccc(F)c1F]

### Step A: Preparation of 3-[(2,3-difluorophenyl)methyl]-3-azabicyclo[3.1.0]hexane-2,4-dione

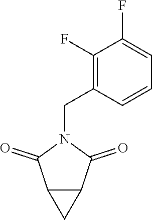 [SMILES: O=C1C2CC2C(=O)N1Cc1cccc(F)c1F]

To a solution of 3-oxabicyclo[3.1.0]hexane-2,4-dione (1.00 g, 8.92 mmol) in polyethylene glycol 400 (2.00 g, 4.71 mmol) was added (2,3-difluorophenyl) methanamine (1.27 g, 8.92 mmol). The reaction was heated to 120° C. for 18 hours before being cooled to room temperature and diluted with EtOAc (20 mL) and water (30 mL). The layers were separated and the aqueous layer was extracted with EtOAc (20 mL) and the combined organic extracts were dried over anhydrous sodium sulfate, filtered, and concentrated under reduced pressure. The crude residue obtained upon concentration was purified by silica gel column chromatography (gradient of 0-60% ethyl acetate in cyclohexane) to afford 3-[(2,3-difluorophenyl)methyl]-3-azabicyclo[3.1.0]hexane-2,4-dione (43%, 900 mg) as a colourless solid.

LC-MS (method 1): Rₜ 1.27, m/z=238 (M+H)⁺.

¹H-NMR (400 MHz, CDCl₃, ppm) δ 7.10 (br d, 1H), 7.06-6.98 (m, 2H), 4.65-4.61 (m, 2H), 2.53 (dd, 2H), 1.56 (td, 1H), 1.40 (dt, 1H).

### Step B: Preparation of 3-[(2,3-difluorophenyl)methyl]-4-hydroxy-3-azabicyclo[3.1.0]hexan-2-one

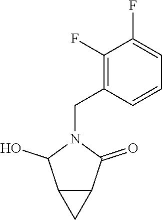 [SMILES: O=C1C2CC2C(O)N1Cc1cccc(F)c1F]

To a stirred solution of 3-[(2,3-difluorophenyl)methyl]-3-azabicyclo[3.1.0]hexane-2,4-dione (300 mg, 1.26 mmol) in methanol (10 mL) was added sodium borohydride (95.7 mg, 2.53 mmol, 2.00 equiv.) at room temperature. The reaction was stirred for 2 hours before being quenched with water (10 mL). EtOAc (20 mL) was added and the layers were separated. The aqueous portion was extracted with EtOAc (20 mL), and the combined organic portions were dried over sodium sulfate, filtered, and concentrated under reduced pressure. The crude residue was purified by silica gel column chromatography (gradient of 0-80% ethyl acetate in cyclohexane) to afford 3-[(2,3-difluorophenyl)methyl]-4-hydroxy-3-azabicyclo[3.1.0]hexan-2-one (93%, 280 mg) as a gummy mass.

LC-MS (method 1): Rₜ 0.94, m/z=240 (M+H)⁺.

¹H-NMR (400 MHz, CDCl₃, ppm) δ 6.98-7.14 (m, 3H), 4.90-4.84 (m, 1H), 4.81-4.59 (m, 1H), 4.38-4.29 (m, 1H), 2.05-1.96 (m, 2H), 1.19-1.09 (m, 1H), 0.56-0.50 (m, 1H).

### Step C: methyl 2-[3-[(2,3-difluorophenyl)methyl]-4-oxo-3-azabicyclo[3.1.0]hexan-2-yl]acetate

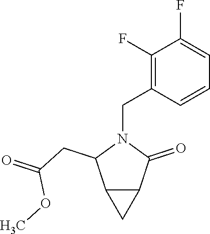 [SMILES: COC(=O)CC1C2CC2C(=O)N1Cc1cccc(F)c1F]

To a solution of methyl diethylphosphonoacetate (1.05 g, 5 mmol) in 1,2-dimethoxyethane (30 mL) at 0° C. was added sodium hydride (200 mg, 5 mmol, 60 mass % in mineral oil). The mixture was stirred for 20 minutes before a solution of 3-[(2,3-difluorophenyl)methyl]-4-hydroxy-3-azabicyclo[3.1.0]hexan-2-one (800 mg, 3.34 mmol) in 1,2-dimethoxyethane (5 mL) was added dropwise. The resulting mixture was slowly warmed to room temperature and stirred for 18 hours before being poured into cold water (15 mL). The mixture was diluted with EtOAc (20 mL) and the layers separated. The aqueous portion was extracted with EtOAc (20 mL), and the combined organic portions were washed with sat. aq. NaCl (50 mL), dried over sodium sulfate, filtered, and concentrated under reduced pressure. The crude residue was purified by silica gel column chromatography (gradient of 0-80% ethyl acetate in cyclohexane) to afford:
methyl 2-[(1R*,2R*,5S*)-3-[(2,3-difluorophenyl)methyl]-4-oxo-3-azabicyclo[3.1.0]hexan-2-yl]acetate (10%, 95 mg) (Compound A.07)

LC-MS (method 1): Rₜ 1.03, m/z=296 (M+H)⁺.

¹H-NMR (400 MHz, CDCl₃, ppm) δ 7.14-7.01 (m, 3H), 4.71-4.65 (m, 1H), 4.19-4.12 (m, 1H), 4.05 (dt, 1H), 3.72 (s, 3H), 2.75 (dd, 1H), 2.30 (dd, 1H), 2.20-2.14 (m, 1H), 2.05 (ddd, 1H), 1.05 (td, 1H), 0.73-0.69 (m, 1H); and
methyl 2-[(1R*,2S*,5S*)-3-[(2,3-difluorophenyl)methyl]-4-oxo-3-azabicyclo[3.1.0]hexan-2-yl]acetate (20%, 210 mg) (Compound A. 10)

LC-MS (method 1): Rₜ 1.03, m/z=296 (M+H)⁺.

¹H-NMR (400 MHz, CDCl₃, ppm) δ 7.14-7.03 (m, 2H), 7.01 (br d, 1H), 4.83 (dd, 1H), 4.11 (d, 1H), 3.74-3.73 (m, 1H), 3.72 (s, 3H), 2.78 (dd, 1H), 2.52 (dd, 1H), 2.00 (d, 1H), 1.81 (td, 1H), 1.11 (dd, 1H), 0.53-0.48 (m, 1H)

### Step D: 2-[(1R*2S*5S*)-3-[(2,3-difluorophenyl)methyl]-4-oxo-3-azabicyclo[3.1.0]hexan-2-yl]acetic acid (Compound A.04)

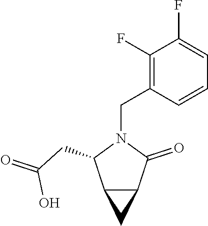 [SMILES: O=C(O)C[C@H]1[C@@H]2C[C@@H]2C(=O)N1Cc1cccc(F)c1F]

To a solution of [methyl 2-[(1R*,2S*,5S*)-3-[(2,3-difluorophenyl)methyl]-4-oxo-3-azabicyclo[3.1.0]hexan-2-yl]acetate] (100 mg, 0.339 mmol) in tetrahydrofuran (4 mL) and water (1 mL) at room temperature was added lithium hydroxide hydrate (17.0 mg, 0.406 mmol). The mixture was stirred for 3 h before water (5 mL) and EtOAc (5 mL) were added. The organic portion was discarded, and the pH of the aqueous portion was adjusted to pH 2 with 1 M aq. HCl and extracted with EtOAc (2×10 mL). The combined organic layers were washed with brine (20 mL), dried over sodium sulfate, filtered, and concentrated under reduced pressure to afford 2-[(1R*,2S*,5S*)-3-[(2,3-difluorophenyl)methyl]-4-oxo-3-azabicyclo[3.1.0]hexan-2-yl]acetic acid (73%, 70 mg) as a colourless solid.

LC-MS (method 1): Rₜ 0.95, m/z=282 (M+H)⁺.

¹H-NMR (400 MHz, DMSO-d₆, ppm) δ 12.47 (s, 1H), 7.35 (d, 1H), 7.21-7.14 (m, 1H), 7.09-7.01 (m, 1H), 4.65 (d, 1H), 4.10 (d, 1H), 3.56 (dd, 1H), 2.78 (dd, 1H), 2.40 (dd, 1H), 1.84 (dd, 1H), 1.79-1.73 (m, 1H), 1.06 (td, 1H), 0.35 (q, 1H)

### Example 2: 2-[(1R*,2R*,5S*)-3-[(2,3,4-trifluorophenyl)methyl]-4-oxo-3-azabicyclo[3.1.0]hexan-2-yl]acetic acid (Compound A.13)

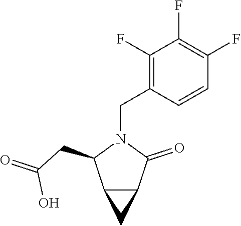 [SMILES: O=C(O)C[C@@H]1[C@@H]2C[C@@H]2C(=O)N1Cc1ccc(F)c(F)c1F]

### Step A: Preparation of ethyl 3-oxo-3-[2-[(2,3,4-trifluorophenyl)methylcarbamoyl]cyclopropyl]propanoate

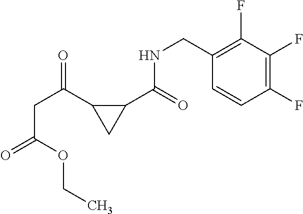 [SMILES: CCOC(=O)CC(=O)C1CC1C(=O)NCc1ccc(F)c(F)c1F]

To a stirred solution of ethyl(2E)-2-(4-oxo-3-oxabicyclo[3.1.0]hexan-2-ylidene)acetate (1.14 g, 5.90 mmol) in toluene (10 mL) was added (2,3,4-trifluorophenyl) methanamine (1.00 g, 5.90 mmol). The resulting mixture was stirred at room temperature for 3 hours then evaporated to dryness. The crude residue was purified by silica gel column chromatography (gradient of ethyl acetate in cyclohexane) to afford ethyl 3-oxo-3-[2-[(2,3,4-trifluorophenyl)methylcarbamoyl]cyclopropyl]propanoate (1.40 g, 65% yield).

LC-MS (method 1): Rₜ 1.18, m/z=344 (M+H)⁺.

¹H-NMR (400 MHz, DMSO-d₆, ppm) δ 1.15 (m, 1H) 1.16-1.25 (m, 3H) 1.41 (td, J=6.60, 4.28 Hz, 1H) 2.15 (td, J=8.77, 6.42 Hz, 1H) 2.24-2.32 (m, 1H) 3.48 (d, J=16.14 Hz, 1H) 3.64 (d, J=16.14 Hz, 1H) 4.01-4.13 (m, 2H) 4.28 (d, J=5.75 Hz, 2H) 7.12-7.19 (m, 1H) 7.23-7.32 (m, 1H) 8.72 (br t, J=5.75 Hz, 1H).

### Step B: Preparation of ethyl(2E)-2-[4-oxo-3-[(2,3,4-trifluorophenyl)methyl]-3-azabicyclo[3.1.0]hexan-2-ylidene]acetate

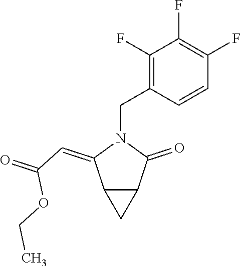 [SMILES: CCOC(=O)/C=C1\C2CC2C(=O)N1Cc1ccc(F)c(F)c1F]

To a stirred solution of 3-ox0-3-[2-[(2,3,4-trifluorophenyl)methylcarbamoyl]cyclopropyl]propanoate (1.40 g, 3.8 mmol) in 1,2-dichloroethane (15 mL) was added p-toluenesulfonic acid (210 mg, 1.2 mmol). The reaction mixture was heated at 90° C. for 4 hours then allowed to cool to room temperature and evaporated to dryness. The crude residue was purified by silica gel column chromatography (gradient of ethyl acetate in cyclohexane) to afford ethyl(2E)-2-[4-oxo-3-[(2,3,4-trifluorophenyl)methyl]-3-azabicyclo[3.1.0]hexan-2-ylidene]acetate (1.10 g, 83% yield).

LC-MS (method 1): Rₜ 1.11, m/z=326 (M+H)⁺.

¹H-NMR (400 MHz, CDCl₃, ppm) δ 1.06 (q, J=3.75 Hz, 1H) 1.23-1.32 (m, 3H) 1.52-1.58 (m, 1H) 2.38 (ddd, J=8.50, 5.07, 3.56 Hz, 1H) 3.60-3.65 (m, 1H) 4.09-4.22 (m, 2H) 4.55 (d, J=16.01 Hz, 1H) 4.68 (d, J=16.01 Hz, 1H) 5.15 (s, 1H) 6.81-6.97 (m, 2H).

### Step C: ethyl 2-[(1R*,2R*,5S*)-3-[(2,3,4-trifluorophenyl)methyl]-4-oxo-3-azabicyclo[3.1.0]hexan-2-yl]acetate (Compound A. 19)

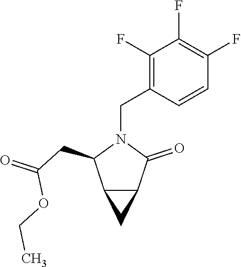 [SMILES: CCOC(=O)C[C@@H]1[C@@H]2C[C@@H]2C(=O)N1Cc1ccc(F)c(F)c1F]

To a solution of ethyl(2E)-2-[4-oxo-3-[(2,3,4-trifluorophenyl)methyl]-3-azabicyclo[3.1.0]hexan-2-ylidene]acetate (200 mg, 0.61 mmol) in dichloromethane (2 mL) at −78° C. was added trifluoroacetic acid (1.42 mL, 18.4 mmol) and triethylsilane (0.92 mL, 6.14 mmol). The reaction mixture was stirred for 10 minutes then refluxed at 40° C. for 7 hours. The reaction mixture was then quenched with aq. NaHCO₃ and extracted three times with ethyl acetate. The combined organics were dried over sodium sulfate then filtered and evaporated to dryness. The crude residue was purified by silica gel column chromatography (gradient of ethyl acetate in cyclohexane) to afford ethyl 2-[(1R*,2R*,5S*)-3-[(2,3,4-trifluorophenyl)methyl]-4-oxo-3-azabicyclo[3.1.0]hexan-2-yl]acetate (152 mg, 76% yield)

LC-MS (method 1): Rₜ 1.05, m/z=328 (M+H)⁺.

¹H-NMR (400 MHz, CDCl₃, ppm) δ 0.67-0.73 (m, 1H) 1.29 (t, J=7.13 Hz, 3H) 2.03 (ddd, J=8.72, 6.03, 3.13 Hz, 1H) 2.13-2.20 (m, 1H) 2.30 (dd, J=15.95, 9.82 Hz, 1H) 2.72 (dd, J=15.95, 4.44 Hz, 1H) 4.01-4.21 (m, 4H) 4.63 (d, J=15.63 Hz, 1H) 6.93-7.06 (m, 2H).

### Step D: 2-[(1R*,2R*,5S*)-3-[(2,3,4-trifluorophenyl)methyl]-4-oxo-3-azabicyclo[3.1.0]hexan-2-yl]acetic acid (Compound A. 13)

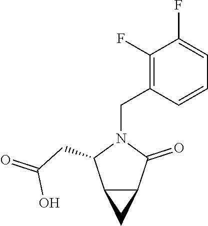 [SMILES: O=C(O)C[C@H]1[C@@H]2C[C@@H]2C(=O)N1Cc1cccc(F)c1F]

To a stirred solution of [ethyl 2-[(1R*,2R*,5S*)-3-[(2,3,4-trifluorophenyl)methyl]-4-oxo-3-azabicyclo[3.1.0]hexan-2-yl]acetate] (100 mg, 0.31 mmol) in methanol (3 mL) at room temperature was added sodium hydroxide (1 mL, 10% aq solution). The mixture was stirred for 3 h. The reaction mixture was then concentrated in vacuo to remove the methanol, then acidified to pH 2 with 2N hydrochloric acid and extracted three times with ethyl acetate. The combined organics were dried over sodium sulfate, filtered and concentrated in vacuo to afford 2-[(1R*,2R*,5S*)-3-[(2,3,4-trifluorophenyl)methyl]-4-oxo-3-azabicyclo[3.1.0]hexan-2-yl]acetic acid (75 mg, 82% yield) as a colourless solid.

LC-MS (method 1): Rₜ 0.93, m/z=300 (M+H)⁺.

¹H-NMR (400 MHz, DMSO-d₆, ppm) δ 0.92-0.99 (m, 1H) 0.96 (td, J=8.10, 4.58 Hz, 1 H) 1.86-1.92 (m, 1H) 2.06-2.12 (m, 1H) 2.23-2.34 (m, 1H) 2.64-2.70 (m, 1H) 3.91 (dt, J=9.60, 4.98 Hz, 1H) 4.09 (d, J=16.02 Hz, 1H) 4.50 (d, J=16.14 Hz, 1H) 7.01-7.08 (m, 1H) 7.25-7.32 (m, 1H) 12.45 (s, 1H)

TABLE 1
Examples of compounds of Formula (I)

| Com- | | | ¹H NMR (400 MHz, |
|---|---|---|---|
| pound | Compound Name | Structure | CDCl₃ unless stated) |
| A.01 | 2-[(1R*,2R*,5S*)-3- [(2,3- difluorophenyl)methyl]- 4-oxo-3- azabicyclo[3.1.0]hexan- 2-yl]acetic acid | 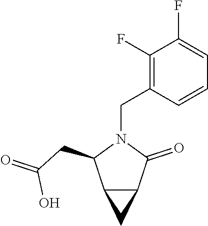 [SMILES: O=C(O)C[C@@H]1[C@@H]2C[C@@H]2C(=O)N1Cc1cccc(F)c1F] | (DMSO-d₆): 12.58-12.33 (s, 1H), 7.45-7.29 (m, 1H), 7.23-7.16 (m, 1H), 7.09- 6.94 (m, 1H), 4.71-4.48 (m, 1H), 4.15-4.08 (m, 1H), 3.96-3.88 (m, 1H), 2.82- 2.63 (m, 1H), 2.42-2.22 (m, 1H), 2.15-2.05 (m, 1H), 1.94-1.72 (m, 1H), 1.10- 0.91 (m, 1H), 0.74 (d, 1H) |
| A.04 | 2-[(1R*,2S*,5S*)-3-[(2,3- difluorophenyl) methyl]- 4-oxo-3- azabicyclo[3.1.0]hexan- 2-yl]acetic acid | 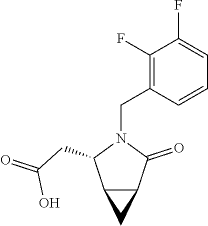 [SMILES: O=C(O)C[C@H]1[C@@H]2C[C@@H]2C(=O)N1Cc1cccc(F)c1F] | (DMSO-d₆): 12.47 (s, 1H), 7.35 (d, 1H), 7.21-7.14 (m, 1H), 7.09-7.01 (m, 1H), 4.65 (d, 1H), 4.10 (d, 1H), 3.56 (dd, 1H), 2.78 (dd, 1H), 2.40 (dd, 1H), 1.84 (dd, 1H), 1.79- 1.73 (m, 1H), 1.06 (td, 1H), 0.35 (q, 1H) |
| A.07 | methyl 2- [(1R*,2R*,5S*)-3-[(2,3- difluorophenyl) methyl]- 4-oxo-3- azabicyclo[3.1.0]hexan- 2-yl]acetate | 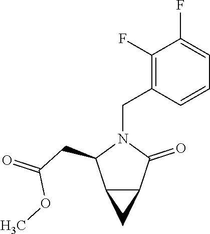 [SMILES: COC(=O)C[C@@H]1[C@@H]2C[C@@H]2C(=O)N1Cc1cccc(F)c1F] | 7.14-7.01 (m, 3H), 4.71- 4.65 (m, 1H), 4.19-4.12 (m, 1H), 4.05 (dt, 1H), 3.72 (s, 3H), 2.75 (dd, 1H), 2.30 (dd, 1H), 2.20-2.14 (m, 1H), 2.05 (ddd, 1H) 1.05 (td, 1H), 0.73- 0.69 (m, 1H) |
| A.10 | methyl 2-[(1R*,2S*,5S*)- 3-[(2,3-difluorophenyl) methyl]-4-oxo-3- azabicyclo[3.1.0]hexan- 2-yl]acetate | 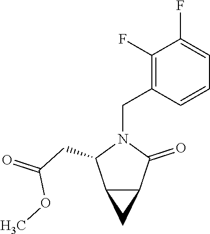 [SMILES: COC(=O)C[C@H]1[C@@H]2C[C@@H]2C(=O)N1Cc1cccc(F)c1F] | 7.14-7.03 (m, 2H), 7.01 (br d, 1H), 4.83 (dd, 1H), 4.11 (d, 1H), 3.74-3.73 (m, 1H), 3.72 (s, 3H), 2.78 (dd, 1H), 2.52 (dd, 1H), 2.00 (d, 1H), 1.81 (td, 1H), 1.11 (dd, 1H), 0.53- 0.48 (m, 1H) |
| A.13 | 2-[(1R*,2R*,5S*)-3- [(2,3,4- trifluorophenyl)methyl]- 4-oxo-3- azabicyclo[3.1.0]hexan- 2-yl]acetic acid | 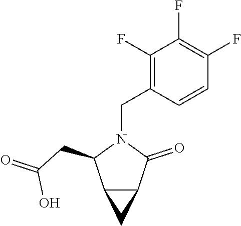 [SMILES: O=C(O)C[C@@H]1[C@@H]2C[C@@H]2C(=O)N1Cc1ccc(F)c(F)c1F] | (DMSO-d₆): 0.92-0.99 (m, 1 H) 0.96 (td, 1 H) 1.86-1.92 (m, 1 H) 2.06-2.12 (m, 1 H) 2.23-2.34 (m, 1 H) 2.64- 2.70 (m, 1 H) 3.91 (dt, 1 H) 4.09 (d, 1 H) 4.50 (d, 1 H) 7.01-7.08 (m, 1 H) 7.25- 7.32 (m, 1 H) 12.45 (s, 1 H) |
| A.61 | 2-[(1R*,2R*,5S*)-3-[(2- fluorophenyl)methyl]-4- oxo-3- azabicyclo[3.1.0]hexan- 2-yl]acetic acid | 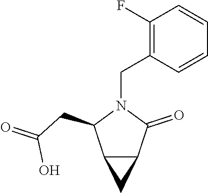 [SMILES: O=C(O)C[C@@H]1[C@@H]2C[C@@H]2C(=O)N1Cc1ccccc1F] | (DMSO-d₆): 0.72 (q, 1 H) 0.96 (td, 1 H) 2.04-2.12 (m, 1 H) 2.23 (dd, 1 H) 2.67 (dd, 1 H) 3.87 (dt, 1 H) 4.05 (d,1 H) 4.52 (d, 1 H), 7.14-7.22 (m, 3 H) 7.29-7.37 (m, 1 H) 12.45 (br s, 1 H) |
| A.64 | 2-[(1R*,2R*,5S*)-3-[(3- fluorophenyl)methyl]-4- oxo-3- azabicyclo[3.1.0]hexan- 2-yl]acetic acid | 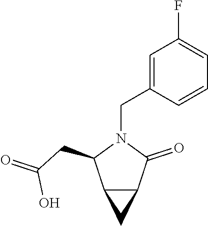 [SMILES: O=C(O)C[C@@H]1[C@@H]2C[C@@H]2C(=O)N1Cc1cccc(F)c1] | (DMSO-d₆): 12.46 (br s, 1 H) 7.34-7.41 (m, 1 H) 7.09 (td, 1 H) 6.97-7.05 (m, 2 H) 4.49 (d, 1 H) 3.99 (d, 1 H) 3.85 (dt, 1 H) 2.71 (dd, 1 H) 1.97- 2.21 (m,2 H) 1.89 (ddd, 1 H) 0.94 (td, 1 H) 0.69 (q, 1 H) |
| A.67 | 2-[(1R*,2R*,5S*)-3-[(4- fluorophenyl)methyl]-4- oxo-3- azabicyclo[3.1.0]hexan- 2-yl]acetic acid | 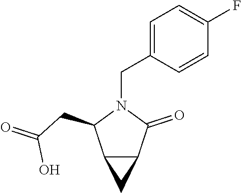 [SMILES: O=C(O)C[C@@H]1[C@@H]2C[C@@H]2C(=O)N1Cc1ccc(F)cc1] | (DMSO-d₆): 12.45 (br s, 1 H) 7.22 (dd, 2 H) 7.15 (t, 2 H) 4.48 (d, 1 H) 3.94 (d, 1 H) 3.80 (dt, 1 H) 2.71 (dd, 1 H) 1.98-2.19 (m, 2 H) 1.84- 1.92 (m, 1 H) 0.93 (td, 1 H) 0.67 (q, 1 H) |
| A.70 | 2-[(1R*,2R*,5S*)-3-[(3- chlorophenyl)methyl]-4- oxo-3- azabicyclo[3.1.0]hexan- 2-yl]acetic acid | 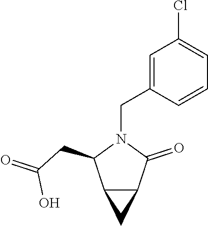 [SMILES: O=C(O)C[C@@H]1[C@@H]2C[C@@H]2C(=O)N1Cc1cccc(Cl)c1] | (DMSO-d₆): 12.46 (s, 1H), 7.30-7.41 (m, 2H), 7.25 (s, 1H), 7.16 (d, 1H), 4.51 (d, 1H), 4.00 (d, 1H), 3.85 (q, 1H), 2.71-2.71 (m, 1H), 2.13- 2.25 (m, 1H), 2.03-2.13 (m, 1H), 1.84-1.98 (m, 1H), 0.90- 1.04 (m, 1H), 0.59-0.75 (m, 1H) |
| A.73 | 2-[(1R*,2R*,5S*)-3- [(3,4- difluorophenyl)methyl]- 4-oxo-3- azabicyclo[3.1.0]hexan- 2-yl]acetic acid | 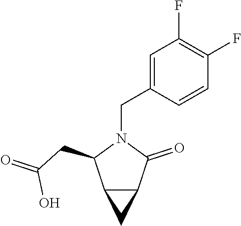 [SMILES: O=C(O)C[C@@H]1[C@@H]2C[C@@H]2C(=O)N1Cc1ccc(F)c(F)c1] | (DMSO-d₆): 0.70 (q, 1 H) 0.95 (td, 1 H) 1.24 (s, 1 H) 2.03-2.10 (m, 1 H) 2.18 (dd, 1 H) 2.69-2.76 (m, 1 H) 3.86 (dt, 1 H) 3.98 (d, 1 H) 4.45 (d, 1 H) 7.04 (br s, 1 H) 7.21-7.28 (m, 1 H) 7.35-7.43 (m, 1 H) 12.44 (br s, 1 H) |
| A.76 | 2-[(1R*,2R*,5S*)-3-[(2- fluoro-4-chloro)methyl]- 4-oxo-3- azabicyclo[3.1.0]hexan- 2-yl]acetic acid | 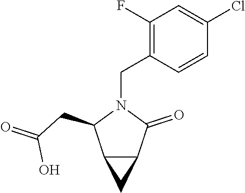 [SMILES: O=C(O)C[C@@H]1[C@@H]2C[C@@H]2C(=O)N1Cc1ccc(Cl)cc1F] | (DMSO-d₆): 12.35 (s, 1 H) 7.42 (dd, 1 H) 7.18-7.25 (m, 2H) 4.03-4.48 (br q, 2 H) 3.86-3.91 (qt, 1H) 2.62-2.67 (br dd, 1 H) 2.21-2.27 (br dd, 1 H) 2.05-2.11 (m, 1 H) 1.86-1.90 (m, 1 H) 0.92- 0.97 (m, 1 H) 0.7-0.73(q, 1 H) |
| A.79 | 2-[(1R*,2R*,5S*)-3-[(3- fluoro-4-chloro)methyl]- 4-oxo-3- azabicyclo[3.1.0]hexan- 2-yl]acetic acid | 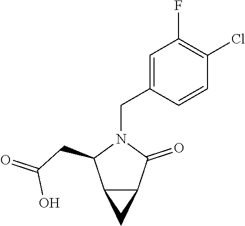 [SMILES: O=C(O)C[C@@H]1[C@@H]2C[C@@H]2C(=O)N1Cc1ccc(Cl)c(F)c1] | (DMSO-d₆): 0.71 (q, 1 H) 0.95 (td, 1 H) 1.87-1.93 (m, 1 H) 2.07 (br d, 1 H) 2.19 (dd, 1 H) 2.71 (dd, 1 H) 3.87 (dt, 1 H) 4.02 (d, 1 H) 4.45 (d, 1 H) 7.05 (br d, 1 H) 7.22 (br d, 1 H) 7.55 (t, 1 H) 12.46 (br s, 1 H) |
| A.81 | 2-[(1R*,2R*,5S*)-3-[(3- chloro-4-fluoro)methyl]- 4-oxo-3- azabicyclo[3.1.0]hexan- 2-yl]acetic acid | 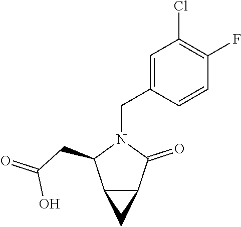 [SMILES: O=C(O)C[C@@H]1[C@@H]2C[C@@H]2C(=O)N1Cc1ccc(F)c(Cl)c1] | (DMSO-d₆): 12.48 (br s, 1 H) 7.35-7.44 (m, 2H) 7.15- 7.25 (m, 1 H) 4.46 (d, 1 H) 3.96 (d, 1 H) 3.85 (dt, 1 H) 2.71-2.79 (m, 1 H) 2.04- 2.22 (m, 2 H) 1.85-1.94 (m, 1 H) 1.20-1.28 (m, 1 H) 0.95 (td, 1 H) 0.69 (m, 1 H) |
| E.01 | 2-[(1R*,3aS*,7aR*)-2- [(2,3- difluorophenyl)methyl]- 3-oxo-3a,4,5,6,7,7a- hexahydro-1H-isoindol- 1-yl]acetic acid | 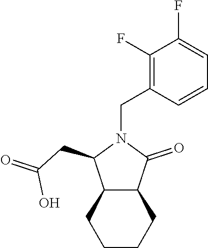 [SMILES: O=C(O)C[C@@H]1[C@@H]2CCCC[C@@H]2C(=O)N1Cc1cccc(F)c1F] | (DMSO-d₆): 0.88-1.01 (m, 2 H) 1.02-1.14 (m, 1 H) 1.37- 1.46 (m, 1 H) 1.47-1.57 (m, 2 H) 1.57-1.70 (m, 1 H) 1.99- 2.10 (m,1 H) 2.28-2.39 (m, 2 H) 2.52-2.62 (m, 2 H) 3.75- 3.90 (m, 1 H) 4.25-4.37 (m, 1 H) 4.59-4.71 (m, 1 H) 6.93- 7.09 (m, 1 H) 7.13-7.25 (m, 1 H) 7.28-7.41 (m, 1 H) 12.32-12.70 (m, 1 H). |
| E.04 | 2-[(1S*,3aS*,7aR*)-2- [(2,3- difluorophenyl)methyl]- 3-oxo-3a,4,5,6,7,7a- hexahydro-1H-isoindol- 1-yl]acetic acid | 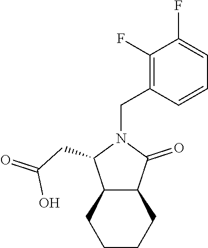 [SMILES: O=C(O)C[C@H]1[C@@H]2CCCC[C@@H]2C(=O)N1Cc1cccc(F)c1F] | (DMSO-d₆): 0.66-0.82 (m, 1 H) 0.83-0.99 (m, 1 H) 0.99- 1.13 (m, 1 H) 1.34-1.51 (m, 3 H) 1.52-1.63 (m, 1 H) 1.91- 1.99 (m,1 H) 2.00-2.10 (m, 1 H) 2.40-2.48 (m, 1 H) 2.57- 2.66 (m, 1 H) 2.66-2.75 (m, 1 H) 3.04-3.18 (m, 1 H) 4.08- 4.24 (m, 1 H) 4.72-4.91 (m, 1 H) 7.10-7.27 (m, 2 H) 7.30- 7.42 (m, 1 H) 12.24-12.57 (m, 1 H) |

### Tables A-E

Specific examples of compounds of Formula (I) are illustrated in the Tables A to E below:

Table A provides 60 (A.01 to A.60) compounds of Formula (I):

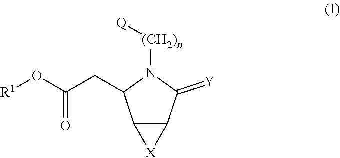

wherein X=CH₂; and wherein the values of Y, n, R¹ and Q, are as defined in Table A below:

TABLE A

| | | | | | Relative | Absolute |
|---|---|---|---|---|---|---|
| | Y | n | R1 | Q | stereochemistry | Stereochemistry |
| A.01 | O | 1 | —H | 2,3-difluorophenyl | Cis- | Rac- |
| A.02 | O | 1 | —H | 2,3-difluorophenyl | Cis- | Ent-1 |
| A.03 | O | 1 | —H | 2,3-difluorophenyl | Cis- | Ent-2 |
| A.04 | O | 1 | —H | 2,3-difluorophenyl | Trans- | Rac- |
| A.05 | O | 1 | —H | 2,3-difluorophenyl | Trans- | Ent-1 |
| A.06 | O | 1 | —H | 2,3-difluorophenyl | Trans- | Ent-2 |
| A.07 | O | 1 | —CH₃ | 2,3-difluorophenyl | Cis- | Rac- |
| A.08 | O | 1 | —CH₃ | 2,3-difluorophenyl | Cis- | Ent-1 |
| A.09 | O | 1 | —CH₃ | 2,3-difluorophenyl | Cis- | Ent-2 |
| A.10 | O | 1 | —CH₃ | 2,3-difluorophenyl | Trans- | Rac- |
| A.11 | O | 1 | —CH₃ | 2,3-difluorophenyl | Trans- | Ent-1 |
| A.12 | O | 1 | —CH₃ | 2,3-difluorophenyl | Trans- | Ent-2 |
| A.13 | O | 1 | —H | 2,3,4-trifluorophenyl | Cis- | Rac- |
| A.14 | O | 1 | —H | 2,3,4-trifluorophenyl | Cis- | Ent-1 |
| A.15 | O | 1 | —H | 2,3,4-trifluorophenyl | Cis- | Ent-2 |
| A.16 | O | 1 | —H | 2,3,4-trifluorophenyl | Trans- | Rac- |
| A.17 | O | 1 | —H | 2,3,4-trifluorophenyl | Trans- | Ent-1 |
| A.18 | O | 1 | —H | 2,3,4-trifluorophenyl | Trans- | Ent-2 |
| A.19 | O | 1 | —CH₃ | 2,3,4-trifluorophenyl | Cis- | Rac- |
| A.20 | O | 1 | —CH₃ | 2,3,4-trifluorophenyl | Cis- | Ent-1 |
| A.21 | O | 1 | —CH₃ | 2,3,4-trifluorophenyl | Cis- | Ent-2 |
| A.22 | O | 1 | —CH₃ | 2,3,4-trifluorophenyl | Trans- | Rac- |
| A.23 | O | 1 | —CH₃ | 2,3,4-trifluorophenyl | Trans- | Ent-1 |
| A.24 | O | 1 | —CH₃ | 2,3,4-trifluorophenyl | Trans- | Ent-2 |
| A.25 | O | 1 | —H | 2,3,5-trifluorophenyl | Cis- | Rac- |
| A.26 | O | 1 | —H | 2,3,5-trifluorophenyl | Cis- | Ent-1 |
| A.27 | O | 1 | —H | 2,3,5-trifluorophenyl | Cis- | Ent-2 |
| A.28 | O | 1 | —H | 2,3,5-trifluorophenyl | Trans- | Rac- |
| A.29 | O | 1 | —H | 2,3,5-trifluorophenyl | Trans- | Ent-1 |
| A.30 | O | 1 | —H | 2,3,5-trifluorophenyl | Trans- | Ent-2 |
| A.31 | O | 1 | —CH₃ | 2,3,5-trifluorophenyl | Cis- | Rac- |
| A.32 | O | 1 | —CH₃ | 2,3,5-trifluorophenyl | Cis- | Ent-1 |
| A.33 | O | 1 | —CH₃ | 2,3,5-trifluorophenyl | Cis- | Ent-2 |
| A.34 | O | 1 | —CH₃ | 2,3,5-trifluorophenyl | Trans- | Rac- |
| A.35 | O | 1 | —CH₃ | 2,3,5-trifluorophenyl | Trans- | Ent-1 |
| A.36 | O | 1 | —CH₃ | 2,3,5-trifluorophenyl | Trans- | Ent-2 |
| A.37 | S | 1 | —H | 2,3-difluorophenyl | Cis- | Rac- |
| A.38 | S | 1 | —H | 2,3-difluorophenyl | Cis- | Ent-1 |
| A.39 | S | 1 | —H | 2,3-difluorophenyl | Cis- | Ent-2 |
| A.40 | S | 1 | —H | 2,3-difluorophenyl | Trans- | Rac- |
| A.41 | S | 1 | —H | 2,3-difluorophenyl | Trans- | Ent-1 |
| A.42 | S | 1 | —H | 2,3-difluorophenyl | Trans- | Ent-2 |
| A.43 | S | 1 | —CH₃ | 2,3-difluorophenyl | Cis- | Rac- |
| A.44 | S | 1 | —CH₃ | 2,3-difluorophenyl | Cis- | Ent-1 |
| A.45 | S | 1 | —CH₃ | 2,3-difluorophenyl | Cis- | Ent-2 |
| A.46 | S | 1 | —CH₃ | 2,3-difluorophenyl | Trans- | Rac- |
| A.47 | S | 1 | —CH₃ | 2,3-difluorophenyl | Trans- | Ent-1 |
| A.48 | S | 1 | —CH₃ | 2,3-difluorophenyl | Trans- | Ent-2 |
| A.49 | O | 2 | —H | 2,3-difluorophenyl | Cis- | Rac- |
| A.50 | O | 2 | —H | 2,3-difluorophenyl | Cis- | Ent-1 |
| A.51 | O | 2 | —H | 2,3-difluorophenyl | Cis- | Ent-2 |
| A.52 | O | 2 | —H | 2,3-difluorophenyl | Trans- | Rac- |
| A.53 | O | 2 | —H | 2,3-difluorophenyl | Trans- | Ent-1 |
| A.54 | O | 2 | —H | 2,3-difluorophenyl | Trans- | Ent-2 |
| A.55 | O | 2 | —CH₃ | 2,3-difluorophenyl | Cis- | Rac- |
| A.56 | O | 2 | —CH₃ | 2,3-difluorophenyl | Cis- | Ent-1 |
| A.57 | O | 2 | —CH₃ | 2,3-difluorophenyl | Cis- | Ent-2 |
| A.58 | O | 2 | —CH₃ | 2,3-difluorophenyl | Trans- | Rac- |
| A.59 | O | 2 | —CH₃ | 2,3-difluorophenyl | Trans- | Ent-1 |
| A.60 | O | 2 | —CH₃ | 2,3-difluorophenyl | Trans- | Ent-2 |
| A.61 | O | 1 | —H | 2-fluorophenyl | Cis- | Rac- |
| A.62 | O | 1 | —H | 2-fluorophenyl | Cis- | Ent-1 |
| A.63 | O | 1 | —H | 2-fluorophenyl | Cis- | Ent-2 |
| A.64 | O | 1 | —H | 3-fluorophenyl | Cis- | Rac- |
| A.65 | O | 1 | —H | 3-fluorophenyl | Cis- | Ent-1 |
| A.66 | O | 1 | —H | 3-fluorophenyl | Cis- | Ent-2 |
| A.67 | O | 1 | —H | 4-fluorophenyl | Cis- | Rac- |
| A.68 | O | 1 | —H | 4-fluorophenyl | Cis- | Ent-1 |
| A.69 | O | 1 | —H | 4-fluorophenyl | Cis- | Ent-2 |
| A.70 | O | 1 | —H | 3-chlorophenyl | Cis- | Rac- |
| A.71 | O | 1 | —H | 3-chlorophenyl | Cis- | Ent-1 |
| A.72 | O | 1 | —H | 3-chlorophenyl | Cis- | Ent-2 |
| A.73 | O | 1 | —H | 3,4-difluorophenyl | Cis- | Rac- |
| A.74 | O | 1 | —H | 3,4-difluorophenyl | Cis- | Ent-1 |
| A.75 | O | 1 | —H | 3,4-difluorophenyl | Cis- | Ent-2 |
| A.76 | O | 1 | —H | 2-fluoro-4-chloro | Cis- | Rac- |
| A.77 | O | 1 | —H | 2-fluoro-4-chloro | Cis- | Ent-1 |
| A.78 | O | 1 | —H | 2-fluoro-4-chloro | Cis- | Ent-2 |
| A.79 | O | 1 | —H | 3-fluoro-4-chloro | Cis- | Rac- |
| A.80 | O | 1 | —H | 3-fluoro-4-chloro | Cis- | Ent-1 |
| A.81 | O | 1 | —H | 3-fluoro-4-chloro | Cis- | Ent-2 |
| A.82 | O | 1 | —H | 3-chloro-4-fluoro | Cis- | Rac- |
| A.83 | O | 1 | —H | 3-chloro-4-fluoro | Cis- | Ent-1 |
| A.84 | O | 1 | —H | 3-chloro-4-fluoro | Cis- | Ent-2 |

Table B provides 84 compounds (B.01 to B.84) of formula (I) wherein X=C(CH₃)₂; and wherein the values of Y, n, R¹ and Q, are defined as in Table A above.

Table C provides 84 compounds (C.01 to C.84) of formula (I) wherein X=CH₂CH₂; and wherein the values of Y, n, R¹ and Q, are defined as in Table A above.

Table D provides 84 compounds (D.01 to D.84) of formula (I) wherein X=CH₂CH₂CH₂; and wherein the values of Y, n, R¹ and Q, are defined as in Table A above.

Table E provides 84 compounds (E.01 to E.84) of formula (I) wherein X=CH₂CH₂CH₂CH₂; and wherein the values of Y, n, R¹ and Q, are defined as in Table A above.

### Biological Examples

Seeds of a variety of test species are sown in standard soil in pots Amaranthus palmeri (AMAPA), Amaranthus retroflexus (AMARE), Setaria faberi (SETFA), Echinochloa crus-galli (ECHCG), Ipomoea hederacea (IPOHE),). After cultivation for one day (pre-emergence) or after 8 days cultivation (post-emergence) under controlled conditions in a glasshouse (at 24/160C, day/night; 14 hours light; 65% humidity), the plants are sprayed with an aqueous spray solution derived from the dissolution of the technical active ingredient in a small amount of acetone and a special solvent and emulsifier mixture referred to as IF50 (11.12% Emulsogen EL360 TM+44.44% N-methylpyrrolidone+44.44% Dowanol DPM glycol ether, which was then diluted to required concentration using 0.2% Genapol XO80 (CAS No. 9043-30-5) in water as the diluent. Compounds are applied at the rates stated. The test plants are then grown in a glasshouse under controlled conditions in a glasshouse (at 24/160C, day/night; 14 hours light; 65% humidity) and watered twice daily. After 13 days for pre and post-emergence, the test is evaluated for the percentage damage caused to the plant. The biological activities are shown in the following table on a five-point scale (5=81-100%; 4=61-80%; 3=41-60%; 2=21-40%; 1=0-20%; NT=not tested).

TABLE B1
Application pre-emergence

| Com- | Rate | | | | | |
|---|---|---|---|---|---|---|
| pound | (g/Ha) | SETFA | ECHCG | AMAPA | AMARE | IPOHE |
| A.01 | 1000 | 5 | 4 | 2 | 4 | 2 |
| A.02 | 1000 | 5 | 5 | 2 | 5 | 2 |
| A.03 | 1000 | 4 | 2 | 1 | 1 | 3 |
| A.04 | 1000 | 3 | 2 | 2 | 2 | 1 |
| A.07 | 1000 | 5 | 4 | 3 | 4 | 2 |
| A.10 | 1000 | 4 | 1 | 2 | 2 | 1 |
| A.13 | 1000 | 5 | 3 | 3 | 4 | 4 |
| A.61 | 1000 | 5 | 3 | 4 | 5 | 1 |
| A.64 | 1000 | 5 | 4 | 4 | 5 | 4 |
| A.67 | 1000 | 3 | 3 | 4 | 3 | 4 |
| A.70 | 1000 | 5 | 4 | 1 | 2 | 1 |
| A.73 | 1000 | 5 | 3 | 3 | 4 | 4 |
| A.76 | 1000 | 3 | 1 | 3 | 1 | 4 |
| A.79 | 1000 | 4 | 2 | 1 | 1 | 1 |
| A.82 | 1000 | 4 | 1 | 2 | 2 | 1 |
| E.01 | 1000 | 1 | 2 | 3 | 3 | NT |
| E.04 | 1000 | 3 | 1 | 4 | 4 | 1 |

TABLE B2
Application post-emergence

| Com- | Rate | | | | | |
|---|---|---|---|---|---|---|
| pound | (g/Ha) | SETFA | ECHCG | AMAPA | AMARE | IPOHE |
| A.01 | 1000 | 5 | 4 | 2 | 4 | 3 |
| A.02 | 1000 | 5 | 5 | 2 | 4 | 1 |
| A.03 | 1000 | 4 | 2 | 1 | 3 | 3 |
| A.04 | 1000 | 4 | 3 | 1 | 1 | 1 |
| A.07 | 1000 | 4 | 3 | 3 | 3 | 3 |
| A.10 | 1000 | 4 | 2 | 1 | 2 | 2 |
| A.13 | 1000 | 5 | 3 | 2 | 3 | 2 |
| A.61 | 1000 | 5 | 3 | 3 | 4 | 3 |
| A.64 | 1000 | 5 | 3 | 3 | 4 | 3 |
| A.67 | 1000 | 4 | 2 | 3 | 1 | 2 |
| A.70 | 1000 | 4 | 3 | 3 | 1 | 1 |
| A.73 | 1000 | 4 | 3 | 1 | 2 | 1 |
| A.76 | 1000 | 3 | 1 | 1 | 1 | 2 |
| A.79 | 1000 | 4 | 1 | 1 | 1 | 1 |
| A.82 | 1000 | 4 | 1 | 1 | 2 | 2 |
| E.01 | 1000 | 1 | 1 | 1 | 1 | 1 |
| E.04 | 1000 | 1 | 1 | 1 | 1 | 1 |

1. A compound of Formula (I) 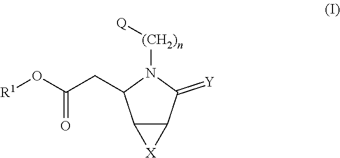
wherein
X=—(CR²R³)ₘ—;
Y=O or S;
Q is phenyl optionally substituted by 1-4 R⁴ substituents;
R¹ is hydrogen or C₁-C₆ alkyl
R² and R³ are independently selected from the group consisting of hydrogen, methyl, fluoro and chloro;
R⁴ is halogen, cyano, C₁-C₆-alkyl, C₁-C₆alkoxy-, C₁-C₆haloalkyl, C₁-C₆-haloalkoxy- and —S(O)ₚC₁-Coalkyl;
m=1, 2, 3 or 4;
n=1 or 2; and
p=0, 1 or 2.

2. A compound according to claim 1, wherein Y is O.

3. A compound according to claim 1, wherein X is —(CR²R³)—.

4. A compound according to claim 2, wherein X is —(CH₂)—.

5. A compound according to claim 1, wherein Q is substituted by one, two or three R⁴.

6. A compound according to claim 1, wherein Q is 2,3 difluorophenyl.

7. A compound according to claim 1, wherein R¹ is hydrogen or methyl.

8. A herbicidal composition comprising a compound according to claim 1 and an agriculturally acceptable formulation adjuvant.

9. A herbicidal composition according to claim 8, further comprising at least one additional pesticide.

10. A herbicidal composition according to claim 9, wherein the additional pesticide is a herbicide or herbicide safener.

11. A method of controlling weeds at a locus comprising application to the locus of a weed controlling amount of a composition according to claim 8.

12. Use of a compound of Formula (I) as defined in claim 1 as a herbicide.
